(19) United States
(12) Patent Application Publication
(10) Pub. No.: US 2009/0151730 A1
(43) Pub. Date: Jun. 18, 2009

(54) INTRAORAL DEVICE

(76) Inventor: Rudiger Hoffknecht, Wurzburg (DE)

Correspondence Address: QUARLES & BRADY LLP, 411 E. WISCONSIN AVENUE, SUITE 2040, MILWAUKEE, WI, 53202-4497, US

(21) Appl. No.: 12/089,760

(22) PCT Filed: Oct. 10, 2006
(86) PCT No.: PCT/DE06/01781

§ 371 Date: Aug. 5, 2008

(30) Foreign Application Priority Data
Oct. 13, 2005 (DE) 10 2005 049 409.9

(51) Int. Cl. A61F 5/56; A61C 5/14

(52) U.S. Cl. 128/848; 128/861

(57) ABSTRACT

Intraoral device (01), in particular for use with sleep-related breathing disorders, having an essentially U-shaped maxillary splint (02), which can be placed on the user's alveolar arch of maxilla, surrounding it in at least some areas, and a mandibular splint (03), which is also essentially U-shaped and can be placed on the user's alveolar arch of mandible, surrounding it in at least some areas, whereby the maxillary splint (02) and the mandibular splint (03) have bite surface areas (06, 07) facing one another, at least one fixation device being provided in their area, with which the mandibular splint (03) can be secured in a predetermined position relative to the maxillary splint (02), and whereby the fixation device comprises at least one fixation element (08) and at least one receptacle element (09) which accommodates the fixation element (08) in at least some areas, whereby the fixation element (08) is provided on the maxillary splint (02) and the receptacle element (09) is provided on the mandibular splint (03) or, conversely, the fixation element (08) is provided on the mandibular splint (03) and the receptacle element (09) is provided on the maxillary splint (02), whereby the fixation element (08) and the receptacle element (09) form an essentially form-fitting plug-type connection.

The present invention relates to an intraoral device, in particular for use in sleep-related breathing disorders, having a maxillary splint designed essentially to be U-shaped, placeable on the user's alveolar arch of maxilla, so that it surrounds the dental arch in at least some areas, and a mandibular splint, which is likewise designed to be essentially U-shaped and can be placed on the user's alveolar arch of mandible to surround same in at least some areas, whereby the maxillary and mandibular splints each have facing bite surface areas, in the area of which at least one fixation device is provided with which the mandibular splint can be secured in a predetermined position relative to the maxillary splint.

Devices of the type defined in the introduction relate to the field of oral orthotic devices which are provided for treatment of snoring and/or sleep apnea in particular. With these diseases, there is a partial or complete blockage of the respiratory tract, usually as a result of the relaxation of the body during sleep. In most persons afflicted with this condition, the occurrence depends on the body position. The patient may thus often experience snoring or apnea when lying in a supine position, when the lower jaw of the sleeper slides dorsally. When the corresponding endogenous sensors detect an oxygen deficiency caused by impaired breathing, a waking reaction occurs, often in combination with a change in position. Patients who snore or have sleep apnea often do not have restorative sleep because the waking reactions prevent the important deep-sleep phases in particular. This leads to fatigue, nervousness and tiredness when awake, even after prolonged sleep.

U.S. Pat. No. 5,642,737 discloses a device designed with a U-shaped maxillary splint and a U-shaped mandibular splint to solve the problem described above, whereby a fixation device in the form of a hook-and-loop tape and/or adhesive closure is provided on the facing bite surface areas of the maxillary and mandibular splints. This device has the aim of securing the maxillary and mandibular splints with respect to one another in a relative position and thereby preventing the mandible from slipping back or being displaced.

However, one disadvantage of the device known from the state of the art is that the fixation device disclosed there ensures only inadequate fixation of the maxillary and mandibular splints relative to one another because during the sleeping phase, considerable forces may act on the maxillary and/or mandibular splint(s) of the person using the device and therefore adequate fixation may not be ensured.

The object of the present invention is therefore to create a device which is especially simple and inexpensive to manufacture on the one hand while also ensuring secure fixation of the maxillary and mandibular splints relative to one another.

This object is achieved by a device having the features of Claim 1.

With the inventive device, the fixation device has at least one fixation element and a receptacle element that receives the fixation element in at least some areas, whereby the fixation element is provided on the maxillary splint and the receptacle element is provided on the mandibular splint or, vice versa, the fixation element is provided on the mandibular splint and the receptacle element is provided on the maxillary splint, whereby the fixation element and the receptacle element form an essentially form-fitting plug-type connection. This has the advantage that a fixation device which ensures an extremely secure seating of the maxillary and mandibular splints relative to one another is created. The position which is predetermined with the help of the fixation device also withstands major displacements of the maxillary halves, e.g., during the sleeping phase, and thus ensures fixation of the predetermined position.

The receptacle element and/or the fixation element itself may be molded directly or indirectly in its splint in the area of the bite surface. It is thus conceivable for the receptacle element and/or the fixation element to be a part of the splint itself and/or an external receptacle element and/or a fixation element and/or a component may be integrated into one splint.

The fixation element may be designed to be essentially conical or mushroom-shaped and may be inserted into the respective receptacle element in the manner of a detachable catch connection. The conical and/or mushroom-shaped design of the fixation element has the advantage that a particularly secure seating of the fixation element in the receptacle element can be ensured.

The secure connection of the two splints relative to one another in the position desired diagnostically/therapeutically, as required for the functioning of the device, may also be implemented in such a manner that it is conceivable to design the plug-type connection essentially as a pushbutton-type connection. Here again, an especially secure seating of the maxillary splint relative to the mandibular splint can also be guaranteed.

According to another embodiment of the invention, at least two, in particular two to ten fixation elements, may be provided on the maxillary and/or mandibular splint. It is thus possible to distribute the maxillary forces that occur in displacement of the two splints in relation to one another uniformly between the maxillary and/or mandibular splints and thus achieve an extremely positive effect for the secure connection and/or fixation of the two splints in relation to one another.

It is possible here for a receptacle element to be assigned each fixation element. However, it is also conceivable for a plurality of receptacle elements to be assigned to each fixation element, in which case the receptacle elements are arranged essentially in a linear arrangement running from the labial area to the pharyngeal area on the respective splint. The device may thus be adapted to different persons and/or different positions of the jaw.

According to a preferred embodiment of the invention, the splint having the receptacle elements has elevations protruding out of the bite surface in some areas with the receptacle elements molded in their surface opposite the respective splint. This has the advantage that unrestricted respiratory function and/or air circulation can be guaranteed for the person wearing the device during the treatment and/or during the adaptation process due to the areas formed between the elevations.

According to another embodiment, at least the areas of the maxillary and/or mandibular splint accommodating the dental arches may essentially be embodied by a formable thermoplastic molding compound, in particular a thermoplastic molding compound that is solid at room temperature, is moderately hard, can be plastified on heating and has a low softening temperature. Adaptation and/or application of the splints is then performed by simply pressing the softenable receptacle areas against the dental arch of the person to be treated to achieve a shape and/or an impression that corresponds exactly to the dental arch of the patient.

According to another especially preferred embodiment of the invention, the maxillary and/or mandibular splints including the fixation and/or receptacle elements can each be manufactured essentially in one piece. This has the advantage that an especially simple and inexpensive production of the device can be achieved. Thus the device may be used first to support diagnostics and therapeutic planning and secondly as a favorable test device for ascertaining the acceptance and tolerability of an intraoral therapeutic device.

According to one embodiment, the thermoplastic molding compound consists essentially of an ethylene-vinyl acetate copolymer, whereby this material is characterized by good tolerability and excellent processability.

A preferred embodiment of the device is explained in greater detail below with reference to the figures, in which:

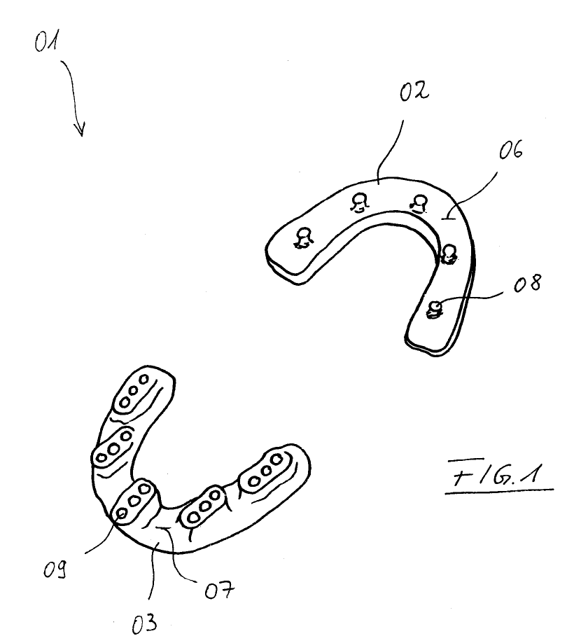

FIG. 1 shows an inventive device comprising a maxillary splint and a mandibular splint in a perspective diagram;

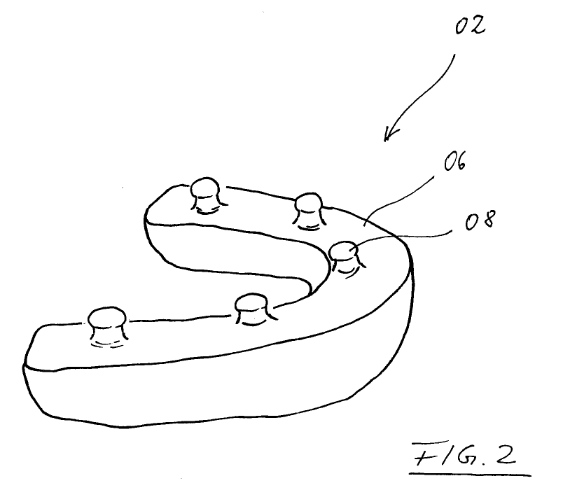

FIG. 2 shows the maxillary splint according to FIG. 1 in an individual diagram;

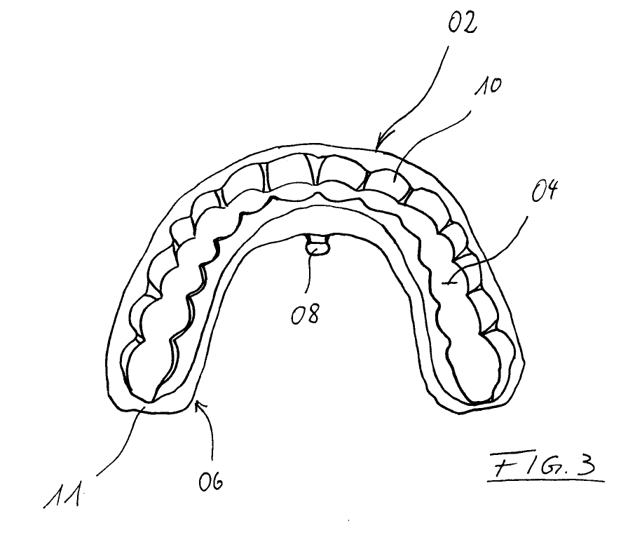

FIG. 3 shows the maxillary splint in a view from beneath;

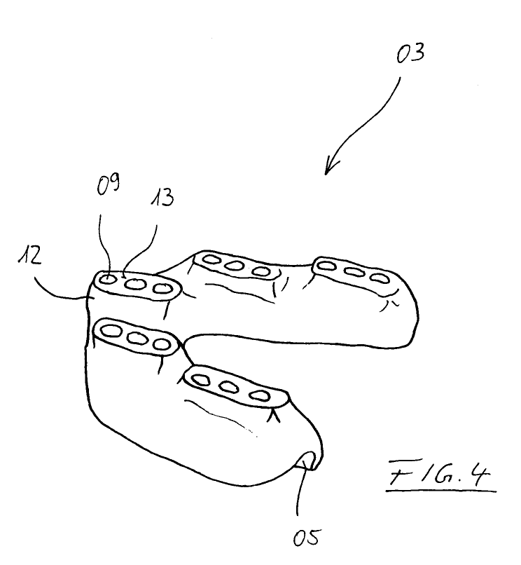

FIG. 4 shows the mandibular splint according to FIG. 1 in an individual view;

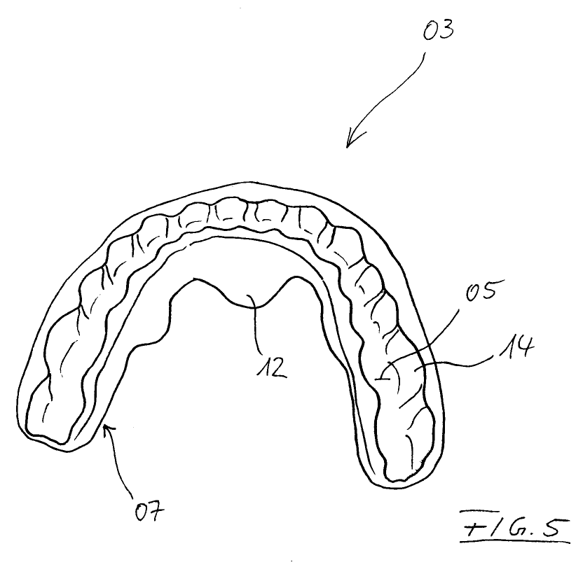

FIG. 5 shows the mandibular splint in a view from beneath.

FIG. 1 shows a device 01 having a maxillary splint 02 and a mandibular splint 03. The two splints are designed to be essentially U-shaped and may each be placed on the user's alveolar arch of the maxilla and/or mandible in at least some areas.

The maxillary splint 02 as well as the mandibular splint 03 have bite surface areas 06, 07 opposite receptacle areas 04, 05 that are not shown in detail or are not clearly discernible, whereby a fixation device is provided on the bite surface areas 06 and 07. A plurality of fixation elements 08 is provided on the maxillary splint 02 and/or on the bite surface area 06, whereby the fixation elements 08 are manufactured here essentially in one piece with the maxillary splint 02 and/or the bite surface area 06.

Receptacle elements 09 are provided on the mandibular splint 03 and/or on the bite surface area 07 of the mandibular splint 03, whereby the receptacle elements 09 are also manufactured essentially in one piece with the mandibular splint 03 and/or with the bite surface area 07. The diagram of the device 01 illustrated here and/or the fixation device provided on the device 01, formed by the fixation element 08 and the receptacle element 09, is illustrated, for example, in such a way that the fixation element 08 is provided on the maxillary splint 02 and the receptacle element 09 is provided on the mandibular splint 03. However, it is also conceivable to design the fixation device in the opposite arrangement, so that the fixation element 08 is provided on the mandibular splint 03 and the receptacle element 09 is provided on the maxillary splint 02. According to this invention, however, this does not make any difference because with the two variants a secure seating can be guaranteed and/or reliable positioning of the two splints in relation to one another can be guaranteed.

The device 01 illustrated here may be used for support and for planning treatment, as a test device for determining acceptance and tolerability of an intraoral device or as a replacement device that is available on a short-term basis when there is a failure of an existing intraoral treatment device. The device 01 is therefore preferably produced essentially from a formable molding compound, in particular from a thermoplastic molding compound that is solid at room temperature, is moderately hard, can be plastified on heating and has a low softening temperature. It is thus possible to adapt the device 01 to the user in the short term, so that it is possible to eliminate a complex adaptation and/or the tedious operation of taking a plaster-like positive mold, for example, as is known from the state of the art. The device 01 may thus function as a simple test device that can be manufactured simply and inexpensively and with which the desired effect of the device can be tested on the user. The device 01 may thus serve in the positive sense, e.g., in the manner of a “disposable item” for one-time usage, that is inexpensive to manufacture and simple to apply, with the help of which the efficacy of a maintenance therapy with a more expensive intraoral device can be predicted.

FIG. 2 shows the maxillary splint 02 according to the device 01 in a detailed view. The maxillary splint 02 having the fixation elements 08 provided on the bite surface area 06 is essentially manufactured in one piece and has a receptacle area (not shown in detail here) which is opposite the bite surface area for the user's dental arch.

The fixation elements 08 themselves are designed essentially in a mushroom shape and may be inserted into the respective receptacle element in the mandibular splint in the manner of a detachable catch connection. With the maxillary splint 02 illustrated here, five fixation elements 08 are provided on the bite surface area 06. At least one receptacle element on the mandibular splint is assigned to each fixation element 08.

Due to the mushroom-shaped design of the fixation elements 08 a very secure and essentially form-fitting plug-type connection with very good holding and/or fixation properties can be guaranteed even when there are major displacement forces acting on the two splints in relation to one another.

FIG. 3 shows the maxillary splint 02 in a view from beneath. This shows clearly the receptacle area 04 into which the user's dental arch can be placed, surrounding it in at least some areas. The receptacle area 04 has prefabricated shapes 10 of various dental forms and can be adapted to the user's dental shape and/or dental arch by applying it and pressing lightly. The receptacle area 04 is preferably designed so that it can be used for a variety of different dental arches and/or dental shapes. However, it is also conceivable to omit a receptacle area 04 and to press the maxillary splint 02 onto the dental arch of the user to the full extent.

In providing a receptacle area 04, an adequate material thickness 11 must be provided between the receptacle area 04 and the bite surface area 06 to ensure a secure seating of the fixation element 08.

FIG. 4 shows the mandibular splint 03 according to the device 01 in a detailed view. The mandibular splint 03 is provided with receptacle elements 09 on its bite surface area 07, whereby a plurality of receptacle elements 09 is assigned to each fixation element. The receptacle elements 09 are arranged essentially in a linear arrangement running from the labial area to the pharyngeal area on the mandibular splint 03.

In addition, the mandibular splint 03 has elevations 12 which protrude out of the bite surface area 07, the receptacle elements 09 being molded in their surface 13 opposite the splint. However, it is also conceivable to shape the receptacle element as an external component, e.g., as a sleeve or the like, and/or to integrate or mold it into the mandibular splint later.

In the embodiment of the lower splint 03 illustrated here, three receptacle elements 09 are provided in each elevation 12. A plurality, namely three here, of receptacle elements 09 is thus assigned to each of the fixation elements 08 on the maxillary splint 02. It is thus possible to implement an adaptation of the position in applying the device in the user's mouth with respect to the seating of the maxillary splint relative to the mandibular splint or vice versa.

The receptacle element 09 is preferably designed so that it corresponds essentially to the shape and/or design of the fixation element 08 which can be inserted into the receptacle element 09. Therefore an essentially form-fitting connection is formed.

FIG. 5 shows the mandibular splint 03 in a view from beneath. This shows clearly the receptacle area 05 which has the prefabricated shapes 14 of different dental forms. The elevations 12 situated on the bite surface area 07 in which the receptacle elements 09 are molded can also be seen clearly.

1. An intraoral device (01), in particular for use with sleep-related breathing disorders, having an essentially U-shaped maxillary splint (02), which can be placed on the user's alveolar arch of maxilla, surrounding it in at least some areas, and a mandibular splint (03) which is also essentially U-shaped and can be placed on the user's alveolar arch of mandible, surrounding it in at least some areas, whereby the maxillary splint (02) and the mandibular splint (03) have bite surface areas (06, 07) facing one another, at least one fixation device being provided in their area with which the mandibular splint (03) can be secured in a predetermined position relative to the maxillary splint (02),
wherein
the fixation device comprises at least one fixation element (08) and at least one receptacle element (09) which accommodates the fixation element (08) in at least some areas, whereby the fixation element (08) is provided on the maxillary splint (02), and the receptacle element (09) is provided on the mandibular splint (03), or conversely, the fixation element (08) is provided on the mandibular splint (03) and the receptacle element (09) is provided on the maxillary splint (02), whereby the fixation element (08) and the receptacle element (09) form an essentially form-fitting plug-type connection.

2. The intraoral device according to claim 1,
characterized in that
the receptacle element (09) and/or the fixation element (08) is/are molded directly or indirectly in its splint in the area of the bite surface (06, 07).

3. The intraoral device according to claim 1, wherein
the fixation element (08) is designed to be essentially conical and/or mushroom-shaped and can be inserted into the respective receptacle element (09) in the manner of a releasable catch connection.

4. The intraoral device according to claim 1,
wherein
the plug-type connection is designed to be essentially a pushbutton-type connection.

5. The intraoral device according to claim 1,
wherein
at least two, in particular two to ten fixation elements (08) are provided.

6. The intraoral device according to claim 1,
wherein
a receptacle element (09) is assigned to each fixation element (08).

7. The intraoral device according to claim 1,
wherein
a plurality of receptacle elements (09) is assigned to each fixation element (08), whereby the receptacle elements (09) are arranged on the splint essentially in a linear arrangement running from the labial area to the pharyngeal area on the respective splint.

8. The intraoral device according to claim 1,
wherein
the splint having the receptacle elements (09) has elevations (12) protruding out of the bite surface (06, 07) in some areas, the receptacle elements (09) being molded in their surface (13) opposite the respective splint.

9. The intraoral device according to claim 1,
wherein
at least the receptacle areas (04, 05) of the maxillary and/or mandibular splints (02, 03) accommodating the dental arches are manufactured essentially from a formable molding compound, in particular from a thermoplastic molding compound that is solid at room temperature, is moderately hard, can be plastified on heating and has a low softening temperature.

10. The intraoral device according to claim 1,
wherein
the maxillary and/or mandibular splints (01, 02) including the fixation elements (08) and/or receptacle elements (09) are each manufactured essentially in one piece.

11. The intraoral device according to claim 9,
wherein
the thermoplastic molding compound is comprised essentially of an ethylene-vinyl acetate copolymer.
